(19) United States
(12) Patent Application Publication
(10) Pub. No.: US 2015/0202055 A1
(43) Pub. Date: Jul. 23, 2015

(54) PROSTHETIC JOINT CUP LINER CUTTING APPARATUS AND METHOD

(71) Applicant: Zimmer, Inc., Warsaw, IN (US)

(72) Inventor: Matthew E. Monaghan, Fort Wayne, IN (US)

(21) Appl. No.: 14/595,774

(22) Filed: Jan. 13, 2015

Related U.S. Application Data
(60) Provisional application No. 61/930,048, filed on Jan. 22, 2014.

(51) Int. Cl. A61F 2/46; B26D 1/08; B26D 7/26; B26D 7/02

(52) U.S. Cl. CPC A61F 2/4612; B26D 7/02; B26D 1/08; B26D 7/2628

(57) ABSTRACT

In some examples, an apparatus is configured to remove a portion of a prosthetic joint cup liner. The apparatus includes a gripping portion sized and shaped to grip the cup liner. A cutting member is adjustable with respect to the gripping portion. The cutting member includes a cutting edge configured to move along a cutting plane to cut the portion of the cup liner to be removed along the cutting plane.

### CLAIM OF PRIORITY

This application claims the benefit of U.S. Provisional Patent Application Ser. No. 61/930,048, filed on Jan. 22, 2014, the benefit of priority of which is claimed hereby, and which is incorporated by reference herein in its entirety.

### BACKGROUND

The present patent application relates to joint arthroplasty, and, more particularly, to an apparatus and method for cutting a prosthetic joint cup liner.

Orthopedic procedures for the replacement of all, or a portion of, a patient's joint have been developed over the last thirty years. Currently, the procedures used to prepare the bone and seat the implants are generally referred to as open procedures. For the purposes of this discussion, the term “open procedure” will refer to a procedure wherein an incision is made through the skin and underlying tissue to fully expose a large portion of the particular joint surface. After the initial incision in the skin, the internal wound can be enlarged to fully expose the areas to be prepared. With the wound enlarged, the clinican can perform various pre-surgical planning tasks prior to commencing a joint restoration procedure.

### OVERVIEW

This overview is intended to provide an overview of subject matter of the present patent application. It is not intended to provide an exclusive or exhaustive explanation of the invention. The detailed description is included to provide further information about the present patent application.

To better illustrate the apparatus and methods disclosed herein, a non-limiting list of examples is provided here:

In Example 1, an apparatus can be configured to remove a portion of a prosthetic joint cup liner, the apparatus comprising: a gripping portion sized and shaped to grip a cup liner; and a cutting portion including: a cutting member movable relative to the gripping portion, the cutting member including a cutting edge configured to move along a cutting plane to cut the portion of the cup liner along the cutting plane; and a cutting plane adjustment member configured to adjust the cutting plane.

In Example 2, the apparatus of Example 1 is optionally configured such that the movement of the cutting plane adjustment member in a first direction moves the cutting portion to increase an angle between the cutting plane and the gripping portion, and movement of the cutting plane adjustment member in a second direction moves the cutting portion to decrease the angle between the cutting plane and the gripping portion.

In Example 3, the apparatus of any one of or any combination of Example 1 or Example 2 is optionally configured such that the cutting plane adjustment member includes a threaded actuator, wherein rotation of the threaded actuator in a first rotational direction increases an angle between the cutting plane and the gripping portion, and rotation of the threaded actuator in a second rotational direction decreases the angle between the cutting plane and the gripping portion.

In Example 4, the apparatus of any one of or any combination of Examples 1-3 is optionally configured such that the cutting plane adjustment member includes an indicator configured to indicate an angle formed between the cutting plane and the gripping portion.

In Example 5, the apparatus of any one of or any combination of Examples 1-4 is optionally configured such that the cutting member includes a cutting member slide configured to move the cutting member along the cutting plane.

In Example 6, the apparatus of any one of or any combination of Examples 1-5 is optionally configured such that the cutting member is a guillotine blade.

In Example 7, the apparatus of any one of or any combination of Examples 1-6 is optionally configured such that the cutting edge is a straight edge, curved edge, or serrated edge.

In Example 8, the apparatus of any one of or any combination of Examples 1-7 is optionally configured such that the cutting member is configured to be replaceable.

In Example 9, the apparatus of any one of or any combination of Examples 1-8 is optionally configured such that the cutting portion includes a support member, configured to receive the cutting member, adjustably coupled to the gripping portion so as to define the cutting plane.

In Example 10, the apparatus of any one of or any combination of Examples 1-9 is optionally configured such that the cutting portion is coupled to the cutting plane adjustment member.

In Example 11, the apparatus of any one of or any combination of Examples 1-10 is optionally configured to include a handle extending from the gripping portion, the handle configured to allow hand-held operation of the apparatus.

In Example 12, the apparatus of any one of or any combination of Examples 1-11 is optionally configured to include a base coupled to the gripping portion, the base configured to support the apparatus on a surface.

In Example 13, the apparatus of any one of or any combination of Examples 1-12 is optionally configured such that the gripping portion includes a first clamping portion pivotably engaged with a second clamping portion, each of the first and second clamping portions configured to engage at least a portion of the cup liner.

In Example 14, the apparatus of any one of or any combination of Examples 1-13 is optionally configured such that the first clamping portion is shaped and sized to accept a top portion of the cup liner, and the second clamping portion is shaped and sized to accept a bottom portion of the cup liner.

In Example 15, the apparatus of any one of or any combination of Examples 1-14 is optionally configured such that the first clamping portion includes at least one retaining member configured to retain the cup liner.

In Example 16, the apparatus of any one of or any combination of Examples 1-15 is optionally configured such that the first clamping member includes a first orifice of a first diameter configured to receive a first portion of the cup liner and the second clamping member includes a second orifice with a second diameter smaller than the first diameter configured to receive a second portion of the cup liner.

In Example 17, a method of removing a portion of a prosthetic joint cup liner with a cutting apparatus having a gripping portion and a cutting portion, the method can comprise: retaining a cup liner, including a high angle portion, at least partially between a first clamping portion of the gripping portion and a second clamping portion of the gripping portion, the first clamping portion being hingedly coupled to the second clamping portion; adjusting an angle of a cutting plane relative to the gripping portion, wherein the cutting portion includes a cutting member configured for slidable movement along the cutting plane; moving a cutting edge of the cutting member along the cutting plane; and removing a portion of the high angle portion of the cup liner, along the cutting plane, with the cutting edge.

In Example 18, the method of Example 17 is optionally configured such that adjusting the angle of the cutting plane includes: moving an adjustment feature of the cutting member in a first direction to increase the angle between the cutting plane and the gripping portion; and moving the adjustment feature in a second direction to decrease the angle between the cutting plane and the gripping portion.

In Example 19, the method of any one of or any combination of Example 17 or 18 is optionally configured such that retaining the cup liner at least partially between the first clamping portion and the second clamping portion includes: placing a top portion of the cup liner within the first clamping portion; and placing a bottom portion of the cup liner within the second clamping portion.

In Example 20, the method of any one of or any combination of Examples 17-19 is optionally configured such that placing the top portion of the cup liner within the first clamping portion includes placing the portion of the cup liner to be removed at a location that intersects with the cutting plane.

In Example 21, the apparatus or method of anyone one of or any combination of Examples 1-20 is optionally configured such that all elements or options recited are available to use or select from.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a liner cutting apparatus in accordance with at least one example.

FIG. 2 is a perspective view of a high angle cup liner in accordance with at least one example.

FIG. 3 is a perspective view of a customized angle cup liner in accordance with at least one example.

FIG. 4 illustrates an example of a method for removing a portion of a joint cup liner in accordance with at least one example.

### DETAILED DESCRIPTION

The present patent application relates to devices and methods for removing a portion of a prosthetic joint cup liner. The device can include a gripping portion sized and shaped to grip a cup liner and a cutting portion including a cutting member adjustably movable with respect to the gripping portion. The cutting member can include a cutting edge configured to move along a cutting plane to cut the portion of the cup liner along the cutting plane. A cutting plane adjustment member can be provided for adjusting the cutting plane of the cutting member.

The cutting device, as described herein, can be used to shape a cup liner, such as a prosthetic cup liner for a shoulder or a hip implant. A cup liner typically fits at least partially within a socket so as to provide a better or customized ball-joint socket fit for the implant. Further, a cup liner can be configured to provide a cushioning layer between a residual limb or natural biological structures and the prosthetic socket or implant. The present cutting device can customize the prosthetic joint cup liner according to patient or surgeon specification. For example, an angle of the prosthetic cup liner can be customized.

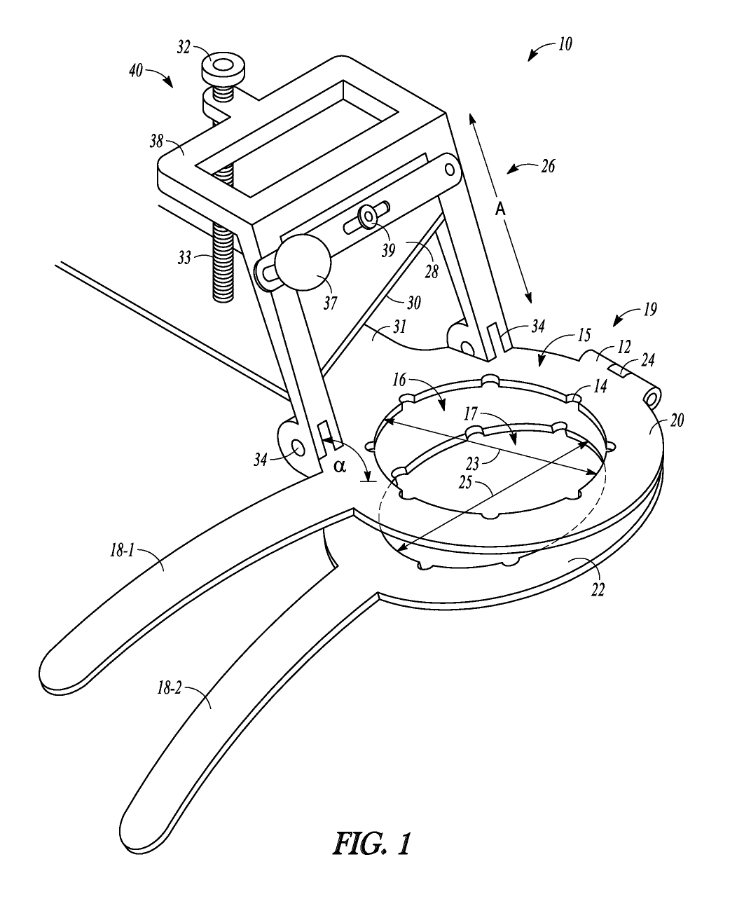

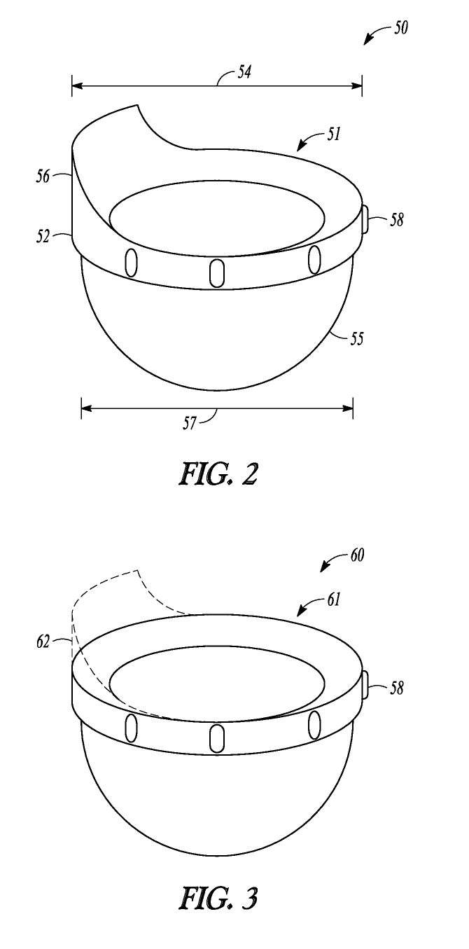

FIG. 1 shows an example of a perspective view of a liner cutting apparatus 10. As shown, the liner cutting apparatus 10 can include a gripping portion 12 and a cutting portion 26. The gripping portion 12 can be configured to retain a prosthetic cup liner, such as a high angle cup liner as described in reference to FIG. 2. The gripping portion 12 can include a first handle 18-1 and a second handle 18-2. Further, the first and second handles 18-1, 18-2 can be hingedly coupled, such as by a gripping portion coupling member 24. The gripping portion coupling member 24 can be configured to couple the first and second handles 18-1, 18-2 at first end 19 of the liner cutting apparatus 10, such that the first and second handles 18-1, 18-2 can be adjusted longitudinally with respect to one another at a second end 21 of the cutting apparatus 10. The first and second handles 18-1, 18-2 can be configured to be gripped, held, partially retained, or the like by a human hand. In an example, the first and second handles 18-1, 18-2 can be contoured so as to provide engagement locations for one or more fingers, a palm, a thumb, or any combination of the foregoing body parts. The first and second handles 18-1, 18-2 can be made of a material to improve grip. In an example, the first and second handles 18-1, 18-2 can be textured so as to improve grip as compared to a smooth surface. Although not shown, the liner cutting apparatus 10 can include a locking mechanism that can allow for retention of the first and second handles 18-1, 18-2 in a prosthetic cup retention position, as described herein.

The first handle 18-1 can include a first orifice 16 of a first diameter 23. The first orifice 16 can be configured so as to at least partially receive the prosthetic cup liner, such as a top portion of the prosthetic cup liner. Further, the first orifice 16 can include at least one restraining member 14. The at least one restraining member 14 can correspond to a size and shape of at least one protrusion of the prosthetic cup liner, as described herein. In an example, the at least one restraining member 14 can be a recess, formed in a wall defining the first orifice, that is configured to restrict rotational or lateral movement of the prosthetic cup liner when the at least one protrusion of the prosthetic cup liner is at least partially received by a corresponding recess. For example, the at least one protrusion of the prosthetic cup liner can slidably engage with a corresponding restraining member 14. The first diameter 23 can be substantially equal to a collar diameter of the prosthetic cup liner, as described herein. Therefore, when the gripping portion 12 is in the prosthetic cup retention position, a non-elevated top surface of the prosthetic cup liner can be substantially flush with a top surface 15 of the gripping portion 12.

The second handle 18-2 can include a second orifice 17 of a second diameter 25. In an example, the second diameter 25 can less than the first diameter 23. Further, the second diameter 25 can be less than the collar diameter of the prosthetic cup liner, as described herein. The first orifice 16 and the second orifice 17 can be configured such that the top portion of the prosthetic cup liner can be at least partially received by the first orifice 16 and a bottom portion of the prosthetic cup liner can be at least partially received by the second orifice, such that the prosthetic cup liner sits within the gripping portion 12.

The gripping portion 12 can be made of a biocompatible material. Suitable biocompatible materials can include, for example, a metallic material such as at least one of a variety of stainless steel composites, titanium, chromium-cobalt, tantalum, or the like, or a non-metallic biocompatible material such as a biocompatible polymer or other plastic material including polyamide, polyphenylsulfone, polyethersulfone, polysulfone, polyketone, polyarylamide, polyether ether ketone (PEEK), polycarbonate, polystyrene, acrylonitrile butadiene styrene (ABS), acrylics, polyetherimide, polyimide, polyphenylsulfone, polymethoylmethacrylate, fiber filled variations of these polymers, amorphous polymeric material, or various other biocompatible polymers.

The cutting portion 26 can be hingedly coupled to the gripping portion 12, such as by at least one cutting portion coupling 34. The cutting portion 26 can include a support member 38. The support member 38 can be adjustably coupled to a cutting plane adjustment member 40 configured to adjust a cutting plane A. In an example, the cutting plane adjustment member 40 can be coupled to an adjustment support member 31 of the gripping portion 12, which can extend from at least one of the first or second handle 18-1, 18-2. In an example, the adjustment support member 31 can be an extension of the top surface 15 of the gripping portion 12. Movement of the cutting plane adjustment member 40 in a first direction (e.g., toward the top surface 15 of the gripping portion 12) can move the cutting portion 26 so as to increase an angle α between the cutting plane A and the gripping portion 12, and movement of the cutting plane adjustment member 40 in a second direction (e.g., away from the top surface 15 of the gripping portion 12) can move the cutting portion 26 so as to decrease the angle α between the cutting plane A and the gripping portion 12.

The cutting plane adjustment member 40 can be coupled to an adjustment rod 33, such that the support member 38 is fixably or removably coupled to the adjustment rod 33. The cutting plane adjustment member 40 can include a threaded actuator 32, wherein rotation of the threaded actuator 32 in a first rotational direction (e.g., clockwise) can increase the angle α between the support member 38 and the gripping portion 12, and wherein rotation of the threaded actuator 32 in a second rotational direction (e.g., counter clockwise) can decrease the angle α between the support member 38 and the gripping portion 12. Turning the adjustment member 32 can, in an example, move the adjustment rod 33 up or down relative to the top surface 15 of the gripping portion 12. That is, turning the adjustment rod 32 can increase or decrease the cutting angle α, wherein the cutting angle α is defined by the angle of the support member 38 relative to the top surface 15 of the gripping portion 12. In an example, the cutting plane adjustment member 40 can include an indicator 41, such as a series of lines or degree markings, configured to indicate the angle α between the cutting plane A and the gripping portion 12.

In an example, the cutting portion 26 can include a cutting member 28 including a cutting edge 30 configured to cut a portion of the prosthetic cup liner along the cutting plane A, such as at the angle α. The cutting member 28 can be slidably engaged with the support member 38, such that the cutting member 28 is movable along the cutting plane A. The cutting member 28 can be slid along the cutting plane A by a cutting member slide 37, adjustably coupled to the cutting member 28 by at least one fastener 39. In an example, the cutting member 28 can be metal, such as stainless steel, or ceramic. The cutting member 28 can be replaceable, such as by undoing the fastener 39. The cutting member 38 can act as a guillotine blade. In various examples, the cutting edge 30 can be at least one of a straight edge, a curved edge, or a serrated edge.

In an example, the liner cutting apparatus 10 can be configured to rest in a base configured to stabilize the apparatus 10 during cutting. Such an example can provide the added benefits of reducing movement of the apparatus and permitting a user to provide additional downward force along the cutting plane A during cutting of the cup liner.

FIG. 2 is a perspective view of a high angle cup liner 50. The high angle cup liner 50 can include a collar 52 having a collar diameter 54, and at least one protrusion 58 extending outwardly from the collar 52 and configured to be received by a corresponding retaining member 14. The collar diameter 54 can be described as the diameter of the collar 52 itself without taking into account the dimensions of the at least one protrusion 58 (i.e., the at least one protrusion 58 is not factored into the collar diameter 54). As described herein, the collar diameter can be greater than the second diameter 25 of the second orifice 17. Further, the collar diameter 54 can be substantially equal to the first diameter 23 of the first orifice 16.

In an example, the at least one protrusion 58 and corresponding retaining member 14 can be configured to align the high angle cup liner 50, such that a high angle portion 56 of the cup liner 50 is adjacent or proximate the cutting portion 26 of the cutting apparatus 10. A bottom portion 55 of the high angle cup liner 50 can have a bottom diameter 57, configured to pass through the first orifice 16 and be at least partially received by the second orifice 17, as described herein.

The high angle portion 56 can include angles, relative to a top surface 51 of the high angle cup liner 50, from about 0.5° to about 80° or more. For example, the high angle portion 56 can include an angle greater than an angle that is generally usable in a prosthetic joint implant, such that a surgeon can remove a selected amount of material from the high angle portion 56 to provide a customized cup liner (FIG. 3) of a desired size and/or shape. That is, the high angle cup portion 56 can be reduced to an angled cup liner commonly used by those skilled in the art.

FIG. 3 is a perspective view of a customized cup liner 60. As used herein, the term “customized” can mean that the cup liner 60 has been cut to a desired size and/or shape with the cutting apparatus 10. A customized angle portion 61 is shown with respect to the original high angle portion 62, which is depicted as a dashed line.

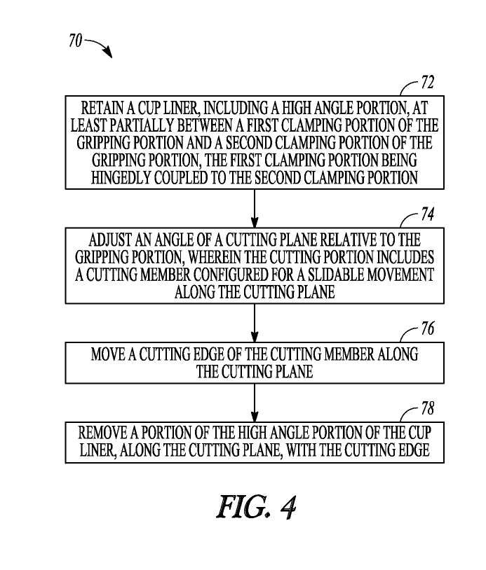

FIG. 4 shows a method 70 for removing a portion of a prosthetic joint cup liner. At 72, the cup liner, including a high angle portion, can be retained with a cutting apparatus. For example, the cup liner can be at least partially retained between a first clamping portion, such a first handle, and a second clamping portion, such as a second handle, wherein the first and second clamping portions are hingedly coupled to one another. A bottom portion of the cup liner can be placed through a first orifice of the first clamping portion and at least partially through a second orifice of the second clamping member. In an example, at least one protrusion on a collar, included on a top portion of the cup liner, can engage a corresponding retaining member of the first orifice, as described herein. Further, the first clamping portion can be moved toward the second clamping portion so as to grip the cup liner between the first clamping portion and the second clamping portion. Moving the first clamping portion can include rotatably moving the first clamping portion with respect to the second clamping portion, the first clamping portion being pivotably coupled to the second clamping portion.

In an example, the first and second clamping portions can be locked together, as described herein. Further, the high angle portion can be configured so as to be disposed above the first clamping portion.

At, 74 a cutting plane angle of a cutting member can be adjusted relative to a top surface of the first clamping portion. In an example, the cutting member can be retained in a support member of the cutting apparatus, wherein the support member is adjustably coupled to a gripping portion of the cutting apparatus so as to define the cutting plane. Adjusting the cutting plane can include adjusting an angle of the cutting plane with respect to the cup liner or the gripping portion. In an example, adjusting the angle of the cutting plane can include moving an adjustment feature of the cutting member in a first direction to increase an angle between the cutting plane and the cup liner. Adjusting the angle of the cutting plane can alternatively or additionally include moving the adjustment feature in a second direction to decrease the angle between the cutting plane and the cup liner.

At 76, a cutting edge of the cutting member can be moved along the cutting plane. As described herein, the cutting member can be slidably engaged with the cutting member. That is, the cutting member can be slidable along the cutting plane and restricted from movement out of the cutting plane by use of a suitable retention member. At 78, a portion of the high angle portion of the cup liner can be removed, along the cutting plane, with the cutting edge. The cutting edge can include any suitable cutting structure or formation suitable for cutting the cup liner, including but not limited to the examples presented herein. In an example, the amount of the high angle portion removed is determined based on the surgeon's discretion.

In an example, the present disclosure can provide a system including the cutting apparatus, as described herein, and at least one high angle cup liner, as also described herein. Further, the system can include various replacement blades, as described herein.

Although the subject matter of the present patent application has been described with reference to various embodiments, workers skilled in the art will recognize that changes can be made in form and detail without departing from the scope of the invention recited in the below claims.

The above Detailed Description includes references to the accompanying drawings, which form a part of the Detailed Description. The drawings show, by way of illustration, specific embodiments in which the present locator apparatus and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

The claimed invention is:

1. An apparatus configured to remove a portion of a prosthetic joint cup liner, the apparatus comprising:
a gripping portion sized and shaped to grip a cup liner; and
a cutting portion including:
a cutting member movable relative to the gripping portion, the cutting member including a cutting edge configured to move along a cutting plane to cut the portion of the cup liner along the cutting plane; and
a cutting plane adjustment member configured to adjust the cutting plane.

2. The apparatus of claim 1, wherein movement of the cutting plane adjustment member in a first direction moves the cutting portion to increase an angle between the cutting plane and the gripping portion, and movement of the cutting plane adjustment member in a second direction moves the cutting portion to decrease the angle between the cutting plane and the gripping portion.

3. The apparatus of claim 1, wherein the cutting plane adjustment member includes a threaded actuator, wherein rotation of the threaded actuator in a first rotational direction increases an angle between the cutting plane and the gripping portion, and rotation of the threaded actuator in a second rotational direction decreases the angle between the cutting plane and the gripping portion.

4. The apparatus of claim 1, wherein the cutting plane adjustment member includes an indicator configured to indicate an angle formed between the cutting plane and the gripping portion.

5. The apparatus of claim 1, wherein the cutting member includes a cutting member slide configured to move the cutting member along the cutting plane.

6. The apparatus of claim 1, wherein the cutting member is a guillotine blade.

7. The apparatus of claim 1, wherein the cutting edge is a straight edge, curved edge, or serrated edge.

8. The apparatus of claim 1, wherein the cutting member is configured to be replaceable.

9. The apparatus of claim 1, wherein the cutting portion includes a support member, configured to receive the cutting member, adjustably coupled to the gripping portion so as to define the cutting plane.

10. The apparatus of claim 9, wherein the cutting portion is coupled to the cutting plane adjustment member.

11. The apparatus of claim 1, further comprising a handle extending from the gripping portion, the handle configured to allow hand-held operation of the apparatus.

12. The apparatus of claim 1, further comprising a base coupled to the gripping portion, the base configured to support the apparatus on a surface.

13. The apparatus of claim 1, wherein the gripping portion includes a first clamping portion pivotably engaged with a second clamping portion, each of the first and second clamping portions configured to engage at least a portion of the cup liner.

14. The apparatus of claim 13, wherein the first clamping portion is shaped and sized to accept a top portion of the cup liner, and the second clamping portion is shaped and sized to accept a bottom portion of the cup liner.

15. The apparatus of claim 13, wherein the first clamping portion includes at least one retaining member configured to retain the cup liner.

16. The apparatus of claim 13, wherein the first clamping member includes a first orifice of a first diameter configured to receive a first portion of the cup liner and the second clamping member includes a second orifice with a second diameter smaller than the first diameter configured to receive a second portion of the cup liner.

17. A method of removing a portion of a prosthetic joint cup liner with a cutting apparatus having a gripping portion and a cutting portion, the method comprising:
retaining a cup liner, including a high angle portion, at least partially between a first clamping portion of the gripping portion and a second clamping portion of the gripping portion, the first clamping portion being hingedly coupled to the second clamping portion;
adjusting an angle of a cutting plane relative to the gripping portion, wherein the cutting portion includes a cutting member configured for slidable movement along the cutting plane;
moving a cutting edge of the cutting member along the cutting plane; and
removing a portion of the high angle portion of the cup liner, along the cutting plane, with the cutting edge.

18. The method of claim 17, wherein adjusting the angle of the cutting plane includes:
moving an adjustment feature of the cutting member in a first direction to increase the angle between the cutting plane and the gripping portion; and
moving the adjustment feature in a second direction to decrease the angle between the cutting plane and the gripping portion.

19. The method of claim 17, wherein retaining the cup liner at least partially between the first clamping portion and the second clamping portion includes:
placing a top portion of the cup liner within the first clamping portion; and
placing a bottom portion of the cup liner within the second clamping portion.

20. The method of claim 19, wherein placing the top portion of the cup liner within the first clamping portion includes placing the portion of the cup liner to be removed at a location that intersects with the cutting plane.
